(19) United States
(12) Patent Application Publication
(10) Pub. No.: US 2009/0036721 A1
(43) Pub. Date: Feb. 5, 2009

(54) Dehydrogenation of ethylbenzene and ethane using mixed metal oxide or sulfated zirconia catalysts to produce styrene

(75) Inventors: Ruozhi Song, Wilmington, DE (US); Anne Mae Gaffney, West Chester, PA (US); Chuen Yuan Yeh, Edison, NJ (US); Philip Jay Angevine, Woodbury, NJ (US)

Correspondence Address: MCCARTER & ENGLISH LLP;CITYPLACE I, 185 ASYLUM STREET, HARTFORD, CT, 06103, US

(73) Assignee: ABB Lummus, Inc.

(21) Appl. No.: 11/888,361

(22) Filed: Jul. 31, 2007

(51) Int. Cl. C07C 15/46

(52) U.S. Cl. 585/438; 585/448; 585/467

(57) ABSTRACT

Methods are described for the simultaneous dehydrogenation of ethylbenzene and ethane in the presence of oxygen or carbon dioxide via a mixed metal oxide (MMO) catalyst or lithium-promoted sulfated zirconia catalyst to prepare styrene monomer from benzene and ethane. An alkylation unit produces ethyl benzene from ethylene and benzene, and an oxydehydrogenation unit produces styrene and ethylene from ethane, ethylbenzene and an oxidizing agent such as oxygen or carbon dioxide. The ethylene produced in the oxydehydrogenation unit is separated and used as feed to the alkylation unit.

### FIELD OF THE INVENTION

This invention relates to catalyst compositions and methods for dehydrogenation of ethylbenzene and ethane for the production of styrene. The catalysts used in the process may be either mixed metal oxides or sulfated zirconia.

### BACKGROUND

Styrene monomer is an important petrochemical used as a raw material for thermoplastic polymer products such as synthetic rubber, ABS resin and polystyrene. Over 90% of the styrene monomer produced today is made by dehydrogenation of ethylbenzene (EB). EB is prepared by the alkylation of benzene, available as a refinery product, with ethylene typically obtained from the cracking or dehydrogenation of ethane.

In the most common commercial process used today, styrene monomer is produced by dehydrogenation of ethylbenzene (EB) in the presence of excess steam over a potassium-promoted iron oxide catalyst. The EB is obtained by alkylating benzene with ethylene. The dehydrogenation step is performed by adding excess steam to EB in an adiabatic reactor under pressurized conditions with a reaction temperature of about 600° C. Although very selective to styrene, this technology has some inherent limitations, including thermodynamic limitations, low conversion rates, required recycling of unconverted reactants, highly endothermic heat of reaction and catalyst deactivation by coking. In this process, the ethylene stream accounts for about 40% of the raw material costs of EB, and superheated steam accounts for an estimated 10% of the cost for styrene production

In an alternative process, as described for example in U.S. Pat. No. 6,031,143 and U.S. Pat. No. 7,002,052, ethane is used as a feedstock in place of ethylene. Ethane is fed with EB to a dehydrogenation unit having a catalyst comprising, for example, gallium and platinum in which a non-oxidative dehydrogenation takes place. Styrene and ethylene are produced in the dehydrogenation unit. The ethylene is recovered and used as a feed to an alkylation unit to produce EB. The dehydrogenation process is typically performed at a temperature of between 450° C. and 700° C., and the conversion of the EB to styrene is relatively low.

In another alternative process, as described in U.S. Publication No. US2005/0070748, EB and ethane are dehydrogenated simultaneously in the presence of oxygen over a mixed metal oxide (MMO) catalyst. The MMO catalyst used in this process may comprise molybdenum, vanadium, niobium and gold. In addition to using a less expensive ethane feedstock, this process is claimed to extend catalyst life due to the less severe operating conditions and the presence of oxygen, which reduces coking. This published application does not describe the conversion rate or selectivity of the process.

Each of these methods suffers from one or more inherent limitations or disadvantages, for example, thermodynamic limitations (i.e., the need for high temperatures), low conversion rate, required cycling of unconverted reactants, high energy input, and catalyst deactivation by coking. As such, there exists an ongoing and unmet need in the industry for less expensive and more efficient methods for styrene production.

### SUMMARY OF THE INVENTION

The present invention relates to an improved process for the production of styrene monomer by the oxidative dehydrogenation (“oxydehydrogenation”) of ethane and ethylbenzene in the presence of a mixed metal oxide (MMO) catalyst, or a sulfated zirconia catalyst. Generally, MMO catalysts are used in processes using oxygen as an oxidizing gas, and a sulfated zirconia catalyst is used when the oxidizing gas is carbon dioxide or a combination of carbon dioxide and oxygen.

In one aspect the invention relates to a catalyst composition for use in the simultaneous dehydrogenation of EB and ethane in the presence of an oxidizing agent or oxidant (i.e., oxidative dehydrogenation). The catalyst is preferably one of: (1) a MMO comprising molybdenum, vanadium, tellurium, niobium and a promoter, (2) a MMO comprising antimony and tin with one or more promoters, or (3) a sulfated zirconia with a lithium promoter.

In another aspect the present invention relates generally to a process for producing styrene using ethane rather than ethylene as a feedstock. The process utilizes an alkylation unit and an oxydehydrogenation (ODH) unit. The process comprises the steps of dehydrogenating ethane and ethylbenzene in the presence of the catalyst in the ODH unit to produce styrene and ethylene. Oxygen or carbon dioxide, or a combination of carbon dioxide and oxygen, may be used as the oxidant in the ODH unit. The ethylene produced in the ODH unit is separated from the styrene and the ethylene is used as a feedstock to an alkylation unit, where the ethylene is combined with benzene under suitable conditions to produce EB. The EB produced in the alkylation unit is sent to the ODH unit. Because the ethylene produced in the ODH unit is used in the alkylation unit, the primary feedstocks required for the overall process are ethane and benzene.

In one embodiment of the invention, an alkylation unit is fed with a stream of benzene and a stream of ethylene. The stream of ethylene is obtained from an oxydehydrogenation unit as described below. The benzene and ethylene are combined in the alkylation unit to form ethylbenzene. The ethylbenzene formed in the alkylation unit is mixed with a stream of ethane and a stream containing an oxidizing agent, and fed to an oxydehydrogenation unit. The dehydrogenation unit contains a catalyst which is capable of catalyzing the simultaneous oxidative dehydrogenation of ethane and ethylbenzene to form ethylene and styrene.

The product stream from the oxydehydrogenation unit is fed to a separation unit to produce a stream containing styrene and a stream containing ethylene. The product stream containing styrene is removed and sent for further processing or packaging. The ethylene stream is fed to the alkylation unit.

A degasifier and a benzene separation unit may be used to separate the ethylbenzene produced in the alkylation unit from unreacted benzene and ethylene. The unreacted benzene and ethylene may be returned to the alkylation unit. A second degasifier may be used to separate the product stream from the dehydrogenation unit.

The catalyst used in the dehydrogenation unit may be a mixed metal oxide catalyst or a sulfated zirconia catalyst. When a mixed metal oxide catalyst is used, the oxidizing agent may be oxygen, which may be provided as air. When the catalyst is a sulfated zirconia catalyst, the oxidizing agent may be carbon dioxide or a mixture of carbon dioxide and oxygen.

The compositions and methods of the present invention result in significant cost savings in chemical feedstock and energy requirements. For example, the process allows the use of ethane rather than ethylene as a feedstock. The oxydehydrogenation process utilizing ethane and ethylbenzene takes place at lower temperatures, reducing or eliminating the need for superheated steam. Energy input is further reduced because of the exothermic nature of the oxidative reaction(s). Furthermore, the process results in higher EB conversion, thereby enabling higher throughput and superior catalyst performance, resulting in higher product yield and longer catalyst life. These advantages are given by way of non-limiting example only, and additional benefits and advantages will be readily apparent to those skilled in the art in view of the description set forth herein.

### BRIEF DESCRIPTION OF THE DRAWINGS

FIG. 1 is a schematic flow chart illustrating an embodiment of the process of the present invention for producing styrene using ethane and benzene as raw materials.

### DETAILED DESCRIPTION OF THE INVENTION

As used herein, “alkylation” generally refers to the reaction of a hydrocarbon, such as an aromatic or a saturated hydrocarbon, with an olefin (e.g., an alkene). As used herein, “promoter” means an accelerator of catalysis, but not a catalyst by itself.

The present invention relates to a process for the simultaneous dehydrogenation of EB and ethane in the presence of an oxidant, for example, oxygen (O₂), carbon dioxide (CO₂) or combinations thereof. The process is referred to as an “oxydehydrogenation” process, or ODH. The process takes place in the presence of a catalyst such as one of the catalysts described in detail below.

In one embodiment of the process, the catalyst used in the process is a mixed metal oxide (MMO). In a preferred embodiment, the MMO catalyst comprises molybdenum (Mo), vanadium (V), tellurium (Te), and niobium (Nb) and one or more promoters, A, selected from the group of Cu, Ta, Sn, Se, W, Ti, Fe, Co, Ni, Cr, Zr, Sb, Bi, Pd, Pt, an alkali metal, an alkaline-earth metal and a rare earth. At least one element selected from Mo, V, Te, and Nb is present in the form of an oxide. In this embodiment of the process, the catalyst comprises the formula: MoVₐTe_{b}Nb_{c}A_{d}Oₓ, where a, b, c, d, and x represent the gram atom ratios of the elements relative to Mo. In preferred embodiments, a, b and c have values lying between about 0.001 and about 4.0, d is between about 0.0001 and about 2.0 and x depends upon the valence of the elements Mo, V, Te and Nb. The composition, structure and method of preparing this catalyst is described in U.S. Patent Publication No. 2005/0085678, the contents of which are hereby incorporated in their entirety.

In another embodiment of the process, the MMO catalyst comprises antimony (Sb), tin (Sn) and oxygen in combination with one or more of the promoters described above. The molar ratio of tin to antimony is generally in the range from about 1:1 to about 20:1. The promoter is present in an amount of between 0.001 and 1.0 relative to the amount of tin in the catalyst. The composition, structure and method of preparing this catalyst is described in detail in U.S. Pat. No. 5,366,822, the contents of which are hereby incorporated in their entirety.

In yet another embodiment, the catalyst comprises sulfated zirconia (Zr) with a lithium promoter. The composition, structure and method of preparing this catalyst are described in detail in Suzuki et al., “Chem. Commun.” 1999, pages 103 to 104, the contents of which are hereby incorporated in their entirety.

Any of the MMO catalyst compositions may be provided on a solid support, for example, silica, alumina, a carbide, titanium oxide, cermet, ceramic, or mixtures thereof. The invention is not limited in this regard, and any appropriate solid support material may be used. In one embodiment, the solid support is present at from about 10% by weight to about 80% by weight with respect to the total weight of the catalyst. In a preferred embodiment in which oxygen is used as the oxidant in the process, a MoVTeNb MMO catalyst of the formula described above is provided on a solid support.

The catalysts used in the process of the present invention can be prepared by conventional methods. For example, the catalysts may be prepared starting from solutions of compounds of the different catalyst components, from solutions of the pure components themselves, or mixtures of both, with the desired atomic ratios. Typically, aqueous solutions of the catalyst components are prepared. Solutions containing the various components of the catalyst may be mixed, the solutions dried to a solid, and the resulting solid may be calcined to produce the desired catalyst. The mixing stage can be done starting from the compounds of the different elements, starting from the actual pure elements in solution, or by hydrothermal methods. The drying stage can be carried out by conventional methods, for example, in a kiln, evaporation with stirring, evaporation in a rotavapor or vacuum drying.

Following drying, the catalyst material may be calcined by conventional methods. For example, the calcination stage of the dry solid can be carried out in an inert gas atmosphere, such as nitrogen, helium, argon or mixtures of these gases, or may be carried out in air or mixtures of air with other gases. The calcination stage can be carried out (a) by flowing inert gas over the catalyst material (with spatial velocities between 1 and 400 h⁻¹) or (b) statically.

In any of the embodiments disclosed herein, the promoter may be of any type generally recognized by those of ordinary skill in the art including, for example, lithium (Li), phosphorus (P), zinc (Zn), copper (Cu), lead (Pb), germanium (Ge), selenium (Se), indium (In), tin (Sn), Ta, Sn, Se, W, Ti, Fe, Co, Ni, Cr, Zr, Sb, Bi, Pd, Pt, an alkali metal, an alkaline-earth metal and a rare earth. The promoter may be added to the catalyst components at the mixing stage and incorporated into the catalyst composition. Alternatively, the promoter may be added to the catalyst material between calcinations steps.

As described above, the MMO catalyst can be supported on a solid such as: silica, alumina, titanium oxide, carbide or mixtures thereof, for example, silicon carbide. In these cases, the fixing of the different elements of the catalyst on the support can be achieved by conventional methods, e.g. incipient wetness, impregnation, excess solution impregnation/ion exchange, or simply by precipitation.

In the process of the present invention, simultaneous dehydrogenation of ethane and ethylbenzene is performed to produce styrene using the catalyst compositions in the presence of an oxidizing agent such as oxygen, carbon dioxide or mixtures thereof. The process of the present invention generally comprises the steps of: a) feeding to an alkylation unit a stream of benzene and a stream of ethylene to form ethylbenzene (EB); b) mixing the EB with a stream of ethane and with a stream of an oxidizing agent, for example, air, oxygen, carbon dioxide or a combination thereof; c) feeding the mixture obtained in b) to an oxydehydrogenation unit containing one of the catalysts described above which results in the simultaneous oxidative dehydrogenation of ethane and ethylbenzene to give ethylene and styrene respectively; d) feeding the product leaving the oxydehydrogenation unit to a separation unit to produce a stream containing styrene and a stream containing ethylene; e) recycling the stream containing ethylene to the alkylation unit.

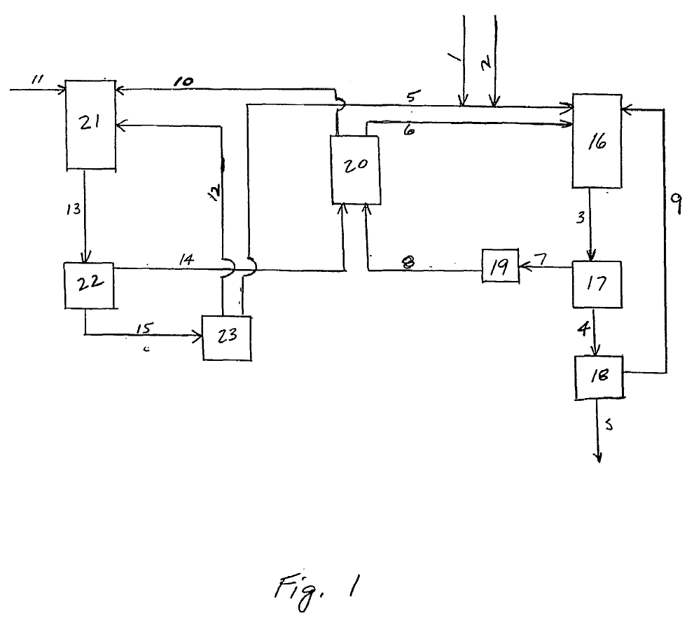

In one embodiment of the process, shown schematically in FIG. 1, the process may be performed as follows. Ethylene produced in the ODH unit 16 as described below is separated in an ethane/ethylene separation unit 20 and fed to the alkylation unit 21 via ethylene feed line 10. Benzene is fed to the alkylation unit 21 via benzene feed line 11. The benzene and ethylene feed streams are fed to the alkylation unit to give a benzene/ethylene molar ratio of preferably between about 2 and 12, more preferably between about 2.5 and 3.5.

The alkylation reaction may be carried out via conventional reactive distillation processes known to those skilled in the art. For example, a zeolite catalyst of the type known for use in alkylation reactions may be used. The alkylation unit is preferably operated at a temperature of between about 150° C. and 350° C., and more preferably between about 190° C. and 230° C., and at a pressure of between about 1-30 bar. If desired, in addition to the reactive distillation column, the alkylation unit may also comprise a fixed bed liquid phase alkylation reactor for treating the products from the reactive distillation column. Although not required, a transalkylation unit to convert diethylbenzene and triethylbenzene to ethylbenzene may also be included.

The ethylene and benzene undergo alkylation in the alkylation unit to produce EB. The product effluent stream from the alkylation unit 21 contains EB and excess ethylene and benzene. The product effluent stream is fed via product line 13 to a degasifier 22 where unreacted ethylene is removed and fed to the ethane/ethylene separation unit 20 via degasifier overhead line 14. The EB and unreacted benzene in the bottoms from the degasifier 22 are fed via bottoms line 15 to a benzene separation unit 23, where the benzene and EB are separated. The benzene is returned to the alkylation unit via benzene return line 12 and the EB is fed to the ODH unit 16 via EB feed line 5. The recovered benzene is optionally dried in a drying column before being recycled to the alkylation unit.

Styrene is produced in an ODH unit 16 by dehydrogenation of ethane and EB. The ODH unit 16 contains one of the catalysts for oxydehydrogenation of ethane and EB as described above, either MMO or sulfated zirconia. The EB produced in the alkylation unit as described above is mixed with ethane from ethane feed line 1 and an oxidant comprising oxygen, carbon dioxide or mixtures thereof from feed line 2. In one preferred embodiment, the ODH unit contains an MMO catalyst and oxygen is used as the oxidizing gas. In another preferred embodiment, sulfated zirconia is used as the catalyst and the oxidizing gas is carbon dioxide or a mixture of carbon dioxide and oxygen. The ODH unit is fed with the mixture of EB, ethane and the oxidant from feed line 5. EB is provided through EB feed line 1 and oxidant gases are provided through feed line 2 to the ODH unit feed line 5. Alternatively, the ODH unit may be fed directly through separate EB, ethane and oxidant feed lines.

If desired, water may be incorporated into the supply to the ODH unit. In the method of oxidative dehydrogenation of ethane to ethylene, an increase is observed in the selectivity of ethylene when the reaction is carried out in the presence of water vapor. The water content in the reaction mixture is preferably between 0% to 80% and more preferably between 20 and 60%.

The EB and ethane are dehydrogenated in the ODH unit 16 to produce a product effluent stream 3 containing ethane, ethylene, styrene and EB. The dehydrogenation reaction is preferably carried out in the gaseous phase in a fixed-bed, a moving-bed or a fluid-bed catalytic reactor. Fluid-bed reactors are preferred for their technological advantages which are well known to those skilled in the field. The reaction temperature is preferably between about 200° C. and 650° C. In one preferred embodiment, the reaction temperature is between about 300° C. and 450° C. using oxygen as the oxidizing gas and an MMO catalyst. In another preferred embodiment, the reaction temperature is between about 500° C. and 650° C. using carbon dioxide as the oxidizing gas and a sulfated zirconia catalyst. The contact time, defined as the ratio between the volume of catalyst and the total flow of supply gases, is preferably between about 0.001 and 100 seconds. Although the contact time depends on the preparation method and composition of the catalyst used, in general it preferably lies between 0.05 and 50 seconds, and more preferably between 0.1 and 25 seconds.

The product stream from the ODH unit is fed to a degasifier 17 through product line 3. In the degasifier 17, the unreacted ethane and ethylene are separated from the unreacted EB and styrene. The overhead stream from the degasifier 17 containing ethane and ethylene is fed via overhead line 7 to a COₓ removal unit 19, which can be of the Selective Olefin Recovery type (SOR), cryogenic type, or any other type. The clean ethane and ethylene are fed via line 8 to an ethane/ethylene separation unit 20. The styrene and EB separated in the degasifier 17 are fed to a separation unit 18 via line 4 where the styrene product is removed via line S. The EB is fed via return line 9 back to the ODH unit 16.

Ethane and ethylene are separated in the ethane/ethylene separation unit 20 and the ethane is fed back to the ODH unit 16 via ethane return line 6, while the ethylene is fed to the alkylation unit 21 via ethylene feed line 10.

The method which gives rise to ethylene is preferably carried out in gaseous phase and in the presence of water vapor. The process results in a longer lasting catalyst, as a consequence of the less severe conditions than in the prior art process, and also the presence of carbon dioxide and/or oxygen reduces coking.

The ethylbenzene product from the alkylation unit is mixed with ethane, which can be entirely fresh ethane or can comprise a mixture of fresh and recycled ethane. To obtain a good balance between the alkylation and dehydrogenation reactions it is preferable for the total ethane, both recycled and fresh, to be present in such an amount as to give molar ratios of ethylbenzene to ethane of between 0.05 and 10, preferably 0.1 and 1. The oxidant may be provided as air, oxygen, carbon dioxide, or a mixture thereof is also introduced as the stream is fed into the oxydehydrogenation (ODH) unit, either as a single stream or at several injection points along the catalyst bed. Recycled ethylbenzene may also be added at this point. Oxidizing agents, for example, oxygen or carbon dioxide, levels are generally 2-20 mol % and more preferably 10-20 mol % in the inlet stream. The oxidizing agents may be introduced in the form of a gas containing molecular oxygen or carbon dioxide or both, which may be air or a gas richer or poorer in molecular oxygen and/or carbon dioxide than air, for example pure oxygen or pure carbon dioxide. A suitable gas may be, for example, oxygen or carbon dioxide or both diluted with a suitable diluent, for example nitrogen or helium.

One skilled in the art will recognize that numerous variations or changes may be made to the process described above without departing from the scope of the present invention. Accordingly, the foregoing description of preferred embodiments is intended to describe the invention in an exemplary, rather than a limiting, sense.

What is claimed is:

1. A process for the production of styrene, comprising the steps of:
a) feeding to an alkylation unit a stream of benzene and a stream of ethylene to form ethylbenzene;
b) feeding the ethylbenzene from the outlet stream of the alkylation unit, a stream of ethane and a stream containing an oxidizing agent to an oxydehydrogenation unit containing a catalyst which is capable of catalyzing the simultaneous oxidative dehydrogenation of ethane and ethylbenzene to form ethylene and styrene;
c) feeding the product leaving the oxydehydrogenation unit to a separation unit to produce a stream containing styrene and a stream containing ethylene; and
d) feeding the stream containing ethylene to the alkylation unit.

2. The process of claim 1, wherein the catalyst is a mixed metal oxide catalyst.

3. The process of claim 1, wherein the catalyst is a sulfated zirconia catalyst.

4. The process of claim 2, wherein the catalyst is a mixed metal oxide having the empirical formula MoVₐTe_{b}Nb_{c}A_{d}Oₓ, wherein A is selected from the group consisting of Cu, Ta, Sn, Se, W, Ti, Fe, Co, Ni, Cr, Zr, Sb, Bi, an alkali metal, an alkaline-earth metal and a rare earth, and wherein a, b and c may be the same or different and have values lying between 0.001 and 4.0, d is between 0.0001 and 2.0 and x depends upon the valence of the elements Mo, V, Te and Nb.

5. The process of claim 2, wherein the catalyst is a mixed metal oxide comprising antimony, tin, oxygen and at least one promoter, wherein the molar ratio of tin to antimony is in the range from about 1:1 to about 20:1.

6. The process of claim 5, wherein the promoter is present in an amount of between 0.001 to 1.0 relative to the amount of tin in the catalyst and the promoter is selected from the group consisting of Li, P, Zn, Cu, Pb, Ge, Se, In, Ta, Se, W, Ti, Fe, Co, Ni, Cr, Zr, Sb, Bi, an alkali metal, an alkaline-earth metal and a rare earth.

7. The process of claim 3, wherein the catalyst is a Li-doped sulfated zirconia.

8. The process of claim 2, wherein the oxidizing agent comprises oxygen.

9. The process of claim 7, wherein the oxidizing agent comprises carbon dioxide or a mixture of carbon dioxide and air.

10. The process according to claim 1, wherein the benzene/ethylene ratio in the alkylation unit is between 2 and 12.

11. The process according to claim 1, wherein in the oxydehydrogenation unit the molar ratio of ethylbenzene to ethane is between 0.05 and 10.

12. The process according to claim 1, wherein the alkylation unit is operated at a temperature of between about 150 and about 350° C. and a pressure of between about 1 and about 30 bar, and the oxydehydrogenation unit is operated at a temperature of between about 200 and 650° C. and a pressure of between about 1 and about 15 bar.

13. The process according to claim 1, further comprising the step of feeding a stream of water vapor to the oxydehydrogenation unit.

14. The process according to claim 1, wherein the stream of ethylene, the stream of ethane and the stream containing an oxidizing agent are mixed prior to being fed to the oxydehydrogenation unit.

15. A process for the production of styrene, comprising the steps of:
a) feeding to an alkylation unit a stream of benzene and a stream of ethylene produced in an oxydehydrogenation unit containing one of a mixed metal oxide or a sulfated zirconia catalyst;
b) feeding the outlet stream from the alkylation unit to a degasifier to separate ethylbenzene from any unreacted benzene or ethylene;
c) feeding the overhead containing ethylene from the degasifier to an ethane/ethylene separation unit;
d) feeding the bottoms containing ethylbenzene and benzene from the degasifier to a benzene separation unit;
e) separating the benzene and ethylbenzene in the benzene separation unit and feeding the benzene to the alkylation unit and the ethylbenzene to the oxydehydrogenation unit containing one of a mixed metal oxide or a sulfated zirconia catalyst;
f) feeding ethane and an oxidizing gas to the oxydehydrogenation unit;
g) feeding the outlet stream from the oxydehydrogenation unit to a degasifier;
h) feeding the overhead containing ethane and ethylene from the degasifier to an ethane/ethylene separation unit;
i) separating the ethane and ethylene in the ethane/ethylene separation unit and feeding the ethane to the oxydehydrogenation unit and the ethylene to the alkylation unit;
j) feeding the bottoms containing ethylbenzene and styrene from the degasifier to a separation unit;
k) separating the ethylbenzene and the styrene in the separation unit and feeding the ethylbenzene to the oxydehydrogenation unit.

16. The process of claim 15, wherein the catalyst is a mixed metal oxide catalyst.

17. The process of claim 15, wherein the catalyst is a sulfated zirconia catalyst.

18. The process of claim 16, wherein the catalyst is a mixed metal oxide having the empirical formula MoVₐTe_{b}Nb_{c}A_{d}Oₓ, wherein A is selected from the group consisting of Cu, Ta, Sn, Se, W, Ti, Fe, Co, Ni, Cr, Zr, Sb, Bi, an alkali metal, an alkaline-earth metal and a rare earth, and wherein a, b and c may be the same or different and have values lying between 0.001 and 4.0, d is between 0.0001 and 2.0 and x depends upon the valence of the elements Mo, V, Te and Nb.

19. The process of claim 16, wherein the catalyst is a mixed metal oxide comprising antimony, tin, oxygen and at least one promoter, wherein the molar ratio of tin to antimony is in the range from about 1:1 to about 20:1.

20. The process of claim 19, wherein the promoter is present in an amount of between 0.001 to 1.0 relative to the amount of tin in the catalyst and the promoter is selected from the group consisting of Li, P, Zn, Cu, Pb, Ge, Se, In, Ta, Se, W, Ti, Fe, Co, Ni, Cr, Zr, Sb, Bi, an alkali metal, an alkaline-earth metal and a rare earth.

21. The process of claim 17, wherein the catalyst is a Li-doped sulfated zirconia.

22. The process of claim 16, wherein the oxidizing agent comprises oxygen.

23. The process of claim 21, wherein the oxidizing agent comprises carbon dioxide or a mixture of carbon dioxide and air.

24. The process according to claim 15, wherein the benzene/ethylene ratio in the alkylation unit is between and 12.

25. The process according to claim 15, wherein in the oxydehydrogenation unit the molar ratio of ethylbenzene to ethane is between 0.05 and 10.

26. The process according to claim 15, wherein the alkylation unit is operated at a temperature of between about 150 and about 350° C. and a pressure of between about 1 and about 30 bar, and the oxydehydrogenation unit is operated at a temperature of between about 200 and 650° C.

27. The process according to claim 15, further comprising the step of feeding a stream of water vapor to the oxydehydrogenation unit.

28. The process according to claim 1, wherein the stream of ethylene, the stream of ethane and the stream containing an oxidizing agent are mixed prior to being fed to the oxydehydrogenation unit.
